(19) United States
(12) Patent Application Publication
(10) Pub. No.: US 2026/0049277 A1
(43) Pub. Date: Feb. 19, 2026

(54) Procedures For Preparing Biological Samples For Overnight Shipment

(71) Applicant: Russell Biotech, Inc., Doylestown, PA (US)

(72) Inventors: Thomas Russell, Doylestown, PA (US); Robert Schmitting, Hillsborough, NC (US)

(73) Assignee: Russell Biotech, Inc., Doylestown, PA (US)

(21) Appl. No.: 19/128,390

(22) PCT Filed: Nov. 9, 2023
(86) PCT No.: PCT/US2023/079192

§ 371 Date: May 8, 2025

Related U.S. Application Data
(60) Provisional application No. 63/425,019, filed on Nov. 14, 2022.

(51) Int. Cl. C12N 5/078; B01L 3/00; C12N 5/00

(52) U.S. Cl. CPC C12N 5/0634; B01L 3/502761; C12N 5/0087; C12N 5/548; B01L 2200/026; B01L 2200/0668; B01L 2200/185

(57) ABSTRACT

Analysis of cell populations for both research and clinical applications such as cell therapy is often performed on whole blood that is close to 24 hours old. The reason for this is that the analysis site is removed from the blood draw site so that blood must be shipped overnight to the analysis site. During the 24-hour shipping period granulocytes, primarily neutrophils, breakdown into cell debris including release of nucleic acids into the blood. Such debris is known to interfere with immunological assays and with rethawing following freezing of samples. There is a need for a method and apparatus to solve this issue. The invention disclosed herein solves the issue. Following blood draw at the draw site the neutrophils are removed using an apparatus that draws blood into a tube that contains anti-CD15 bound to nickel magnetic particles. Following mixing the tube is placed in a magnetic field and then while still in the magnetic field the blood with neutrophils removed is transferred into a new tube for shipment to the analysis site.

### CROSS REFERENCE

This application is the US national application of PCT/US2023/079192, filed 9 Nov. 2023, and which claims benefit of and priority to U.S. Provisional Patent Application No. 63/425,019 filed 14 Nov. 2022, now expired, the disclosure of which is incorporated by reference in its entirety.

### BACKGROUND OF THE INVENTION

Field of the Invention: This invention relates broadly to the field of sample preservation prior to shipment to a site for analysis or clinical applications. More specifically the invention relates to the use of solid phase dense, metallic magnetic particles in combination with a transfer device to very rapidly remove granulocytes and/or platelets, known to adversely affect sample quality, from undiluted whole blood prior to shipment. The invention includes both means and apparatus that permit removal of the desired cells at the blood draw site prior to shipment.

### DESCRIPTION OF RELATED ART

Immunological monitoring is a critical component in both basic research and clinical research especially for studies related to cell therapy/transplantation. The majority of samples for immunological monitoring are derived from mononuclear cells (PBMC) within whole blood (WB). Typically, the WB samples are drawn into a vacutainer blood collection tube and the blood is processed to remove red blood cells and granulocytes, most commonly via Ficoll gradient separation. This process is somewhat labor intensive requiring dedicated laboratory space, equipment, and trained lab personnel.

More often, the only solution is to ship the WB to a processing center for PBMC isolation and freezing, where the immune monitoring occurs at a later point in time. Also, freeze/thaw process often leads to significant cell losses and decreased viability. Functional integrity of the peripheral blood mononuclear cells (PBMC) declines with time from blood draw (1, 2), and it is well established that granulocytes play a major role in this functional decline (3, 4). The longer the storage time until processing of whole blood, the higher is the contamination degree of PBMC with granulocytes which adversely affect immunological functional studies i.e. using ELISPOT and intracellular cytokine analysis by Flow Cytometry. During prolonged storage granulocytes become activated and change their buoyancy profile that leads to their less efficient separation during isolation on Ficoll.

Activated granulocytes are known to suppress T-cell function by down modulating the signal transducing zeta chain of the CD3 molecule (5). Therefore, depletion of granulocytes and/or procedures that will lead to effective granulocyte removal and inhibition of their activation and its inhibitory effects on T-cells shortly after blood draw would extend the functional integrity of PBMC samples to provide meaningful clinical immunological monitoring data. An easy to integrate solution at the blood draw site with a controllable and minimal work load is desirable. Can magnetic bead technology solve this issue? Though magnetic cell separation technology has been around for decades and a number of manufactures including Miltenyi Biotec (8) and ThermoFisher/DYNAL (9), but not limited to, offer magnetic particles that can remove granulocytes, their procedures will not solve the problem at hand. Why: these procedures, as detailed below, do not lend themselves to rapid removal of granulocytes directly in un-diluted whole blood an absolute requirement for removal of granulocytes at the blood collection site.

The ThermoFisher website states the following: “Dynabeads can be added directly to undiluted blood if reduced cell isolation efficiency is tolerated . . . .” This is not desirable. Also “When incubating Dynabeads and cells, the incubation temperature must be 2-8° C. to reduce phagocytic activity and other metabolic processes. The magnetic beads need to be incubated for 30 min (depletion) at 2-8° C. with gentle tilting and rotation”.

The Miltenyi Biotec website states the following: “CD15 MicroBeads were developed for depletion of CD15 positive cells from human lysed peripheral blood” For shipping whole blood lysing of the red cells is not an option and cannot be done conveniently at the blood draw site. Also, the Miltenyi process requires columns for depletion of cells which results in significant loss of desired cells (PBMCs).

The magnetic particles of the art based on non-metallic iron oxide magnetic material simply are not suited for use at a blood draw site.

There is a definite need for methods and devices that yield granulocyte free or granulocyte plus platelet free whole blood at the blood draw site rapidly and conveniently. The inventions(s) disclosed herein solve this problem yielding whole blood devoid of granulocytes or granulocytes and platelets immediately ready for shipment in a timely and convenient fashion.

### SUMMARY OF THE INVENTION

The invention disclosed herein provides methods and means (apparatus) to sterilely remove granulocytes or granulocytes plus platelets at the blood draw site i.e. Lab Corp or Quest, but not limited to. One embodiment of the invention includes the use of dense, metallic, magnetic beads coupled to anti-granulocyte surface molecules (CD15) or anti-granulocyte surface molecules (CD15) and anti-platelet surface molecules (CD41 and/or CD61) that effectively bind to granulocytes or granulocytes plus platelets very rapidly in undiluted whole blood as described in U.S. Pat. No. 9,435,799 incorporated here in its entirety by reference. In one preferred embodiment, the granulocytes are then removed from the solution by magnetic separation. This is all accomplished in a transfer device as disclosed herein. Blood from a blood draw tube is placed in the transfer device and punctured by a needle. The vacutainer draws blood from the blood draw tube into a new tube containing the desired magnetic particles. The tube is removed, mixed by end-over-end mixing for the appropriate time. The tube is then placed in a magnetic separation device that separates the CD15 bound granulocytes from the undiluted blood. The tube in the magnetic field is then placed in a second transfer device that punctures the tube and transfers the blood depleted of granulocytes into a new sealed tube that is ready for shipment to an external site.

In another embodiment, the particle bound granulocytes are removed by gravity separation due to the density of the particles.

### BRIEF DESCRIPTIONS OF DRAWINGS

FIG. 1. High cell recovery of non-targeted cell populations. The top figure is the light scatter histogram of PBMCs run on a Becton Dickinson Flow Cytometer. Y-axis: side light scatter (90 degree); X-axis: forward light scatter. Three cell populations are easily distinguished: Top: granulocytes; Middle: monocytes; Bottom: lymphocytes. The bottom figure represents the light scatter histogram of PBMCs following the depletion of granulocytes using CD15 nickel particles using the method described herein.

FIG. 2. Rapid removal of granulocytes using anti-CD15 metallic, magnetic particles. Whole blood was analyzed on a Beckman-Coulter 3-part differential analyzer. Three cell populations can be distinguished: Top left figure (Control): Left: lymphocytes; Middle: monocytes; Right: granulocytes. The top right, bottom left and bottom right figures demonstrate the removal of CD15 positive granulocytes using the method described herein as a function of incubation time with the CD15 metallic, magnetic particles. Incubation time ranged from 1 minute to 3 seconds.

FIG. 3. Effect of CD15 depletion on various cell populations present in whole blood. Anti-CD15 nickel particles of the invention were used to deplete granulocytes and the effect of the depletion on lymphocyte subset populations (CD3, CD4 and CD8) was examined.

FIG. 4. Illustration of transfer device

The inner diameter dimension is about 2 mm greater in diameter than blood drawing tube septum diameter which allows for free travel of the blood tube into the transfer device. The outer diameter is such that the wall thickness provides rigidity to the transfer device. The needle is centered in the diameter of the device and protrudes from both top and bottom a distance that allows full penetration through the septum cap into the blood tube. The entire length of the device (bottom to top) is dictated by a combination of the length of the needle necessary to pass through the blood tube septum cap of both tubes in the transfer process, and the center support section that holds the needle. The center support section holding the needle is about 5 mm thick and the device wall is about 4 mm thick.

FIG. 5. Illustration of another transfer device. Commonly used vacutainer transfer devices are available with internal needle and male or female luer-lock adaptor systems. These devices may be coupled by joining a male to female devices, coupling male to male using female-female adapter, or female to female using a male-male adaptor such that two devices are coupled to allow the passage of blood from one vacutainer to another vacutainer.

### DETAILED DESCRIPTION OF PREFERRED EMBODIMENTS

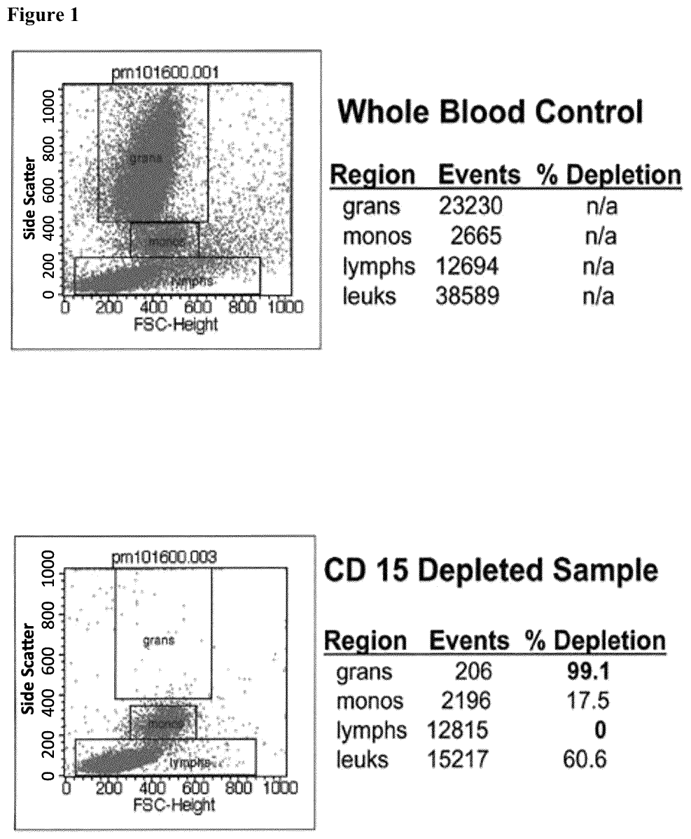

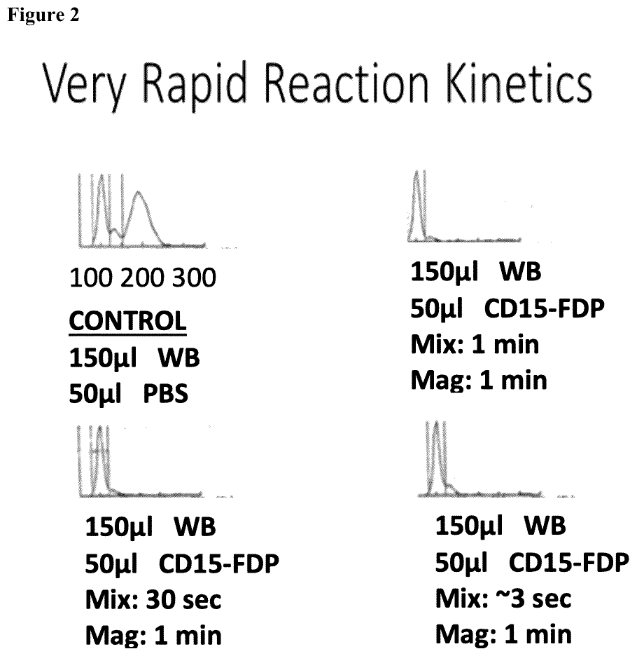

In order to provide granulocyte free blood for overnight shipment a procedure is needed that can be performed at the blood draw site with minimal involvement by laboratory personnel. In a preferred embodiment, dense, metallic, magnetic particles that meet this requirement exist as described in U.S. Pat. No. 9,435,799 incorporated herein by reference. The preferred metallic magnetic particle is composed of nickel metal often with a thin nickel oxide layer on the surface as described in U.S. Pat. No. 9,435,799. These particles meet all requirements for working at a blood draw site: they work directly in undiluted whole blood, do not bind or trap cells of interest yielding close to 100% of recovery of non-targeted cells in this case PBMC (FIG. 1) and the particles bind to the targeted cells rapidly on the order of seconds to minutes (FIG. 2).

The optimum bead size, reaction time and magnetic separation time is determined by any means known in the art and described in U.S. Pat. No. 9,435,799 using the transfer device disclosed herein.

The best bead size will be determined using particles in the size range from about 0.5 micron to 3.5 micron but not limited thereto. For magnetic separation, the beads tested will be as described in U.S. Pat. No. 9,435,799 with a preferable bead composed of nickel metal but not limited thereto. Any magnetic metal bead is considered in the present disclosure. The magnetic nickel bead can be manufactured as disclosed in U.S. Pat. No. 9,435,799 or obtained commercially from sources such as Sigma or Novamet.

The density of the particles can be in the range of 4-10 g/cc with a preferred density of around 9 g/cc.

For removal of granulocytes from undiluted whole blood anti-CD15 antibodies will be coupled to the magnetic bead by means known in the art including direct adsorption or covalent coupling. Though anti-CD15 monoclonal antibody is preferred any monoclonal or polyclonal antibody bound to the metallic magnetic bead that removes granulocytes is included in the disclosure.

The preferred embodiment as disclosed above may also include beads that remove platelets which are known to be sticky and therefore may also need to be removed at the blood draw site to provide the best material for overnight shipping. Experiments will be performed using the transfer device disclosed herein to determine if removal of granulocytes is sufficient or whether removal of granulocytes and platelets is the desired means to prepare the whole blood sample for shipment.

The manual method for removal of granulocytes is summarized here:

The transfer device disclosed herein will carry out the procedure without need for the user to add beads or blood manually making the procedure convenient for use at blood draw sites prior to shipment. For the purpose of clarification, the anti-CD15 and/or anti-platelet magnetic particles i.e. CD41 and/or CD61 will be in a vacutainer tube. The beads in the vacutainer tube can be in a liquid state or a lyophilized state. Once the blood has been transferred to the vacutainer tube the manual method as described here:

### Method:

1. Using the transfer device transfer the WB from a vacutainer tube into the vacutainer tube containing the magnetic particles
2. Remove the tube containing the whole blood and magnetic particles from the transfer device
3.Mix on end-over-end mixer* for up to 5 minutes (actual time may vary and the best time will be determined experimentally)
4.Place tube in magnetic field for 1 minute (actual magnetic separation time may vary and will be determined experimentally)
5.Using the second transfer device while tube is still in the magnetic field, transfer the granulocyte and/or granulocyte plus platelet depleted blood into a new vacutainer tube.
6. Prepare the vacutainer tube for shipment

### Equipment Required:

*Mixer: Due to the approximately 5-fold difference in density between CD15 particles and cells, proper mixing is essential to ensure contact between the particles and the targeted cells. Mixing is accomplished by end-over-end mixing using i.e. an ATR Rotomix mixer with variable speed. Recommended mixing speed is 15-30 rpm.

### Magnetic Separation:

a. ideal magnets for use with anti-CD15 magnetic particles disclosed herein can be obtained from Dexter Magnetic Technologies. Different magnets are available for sample volumes from approximately 0.5 mL to 50 mL
b. Magnets from suppliers of superparamagnetic particles will also work as long as while placed in the magnetic field the blood tube can be placed in the second transfer device
c. Manufactures of magnetic separation devices such as Life Sep, but not limited thereto, can by means known in the art manufacture a magnetic separation device that is compatible with the transfer device disclosed herein.

In another embodiment, dense particles that settle by gravity as disclosed in U.S. Pat. Nos. 5,576,185 and 9,435,799 can be used in the transfer device disclosed herein. The particles are dense (4-10 g/cc) and can be composed of magnetic or non-magnetic material. An advantage of magnetic particles is simply that following gravity settling a magnet can be placed at the bottom of the tube to hold the particles in place prior to transfer.

### Detailed Description of the Transfer Device:

The particles used to deplete granulocytes or granulocytes and platelets by magnetic or gravity separation will be placed in a blood draw tube under vacuum. The particles can be in a liquid state (preferred volume to be determined by normal experimentation) or lyophilized by normal lyophilization procedures known in the art.

### The Transfer Device Will Now be Described in Detail:

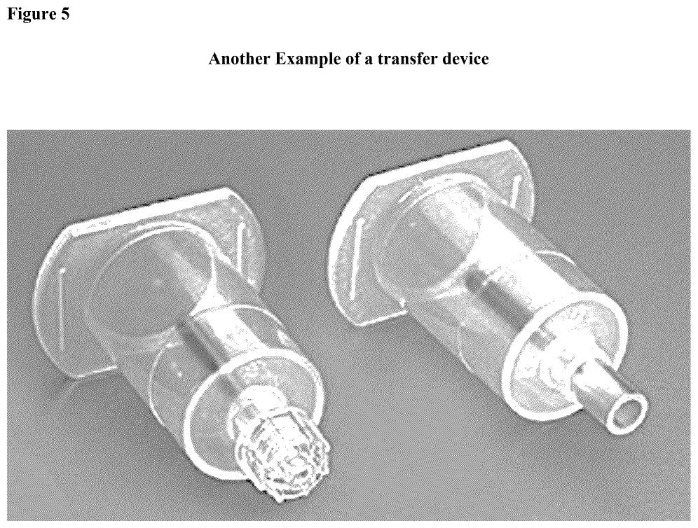

The transfer device is utilized in the transfer of one tube under atmospheric pressure containing material (liquid) to be transferred into another tube under less pressure (vacuum) FIGS. 4 and 5 are examples of transfer devices covered by this invention. Any device that meets the requirements of a transfer device as disclosed herein is anticipated by this disclosure. In the normal course of action, the transfer device is placed on top of a freshly drawn tube of blood and applying enough pressure to cause the needle to puncture the septum cap. The blood tube with the attached transfer device is inverted and placed onto an evacuated blood tube containing the antibody labeled magnetic particles (bead tube). The blood tube, transfer device, and bead tube are now connected so that blood is drawn into the evacuated bead tube through the needle which has punctured both blood tube and bead tube. The empty blood tube and transfer device is removed and discarded. The bead tube containing the transferred blood product is placed on an appropriate end-over-end mixer and mixed for the appropriate time. The bead tube containing blood is removed from the mixer and placed on an appropriate magnetic holder (Stemcell, catalog #18001, or equivalent) and mixed by alternate inversions to allow the magnetic bead bound targets to be magnetically attracted to the magnetic field within the tube. After the appropriate time a fresh transfer device is placed on top the bead/blood tube while still attached to the magnetic holder. Any magnetic device is appropriate as long as the magnetic device does not interfere with the ability of the tube to be placed in the second transfer device. Once the transfer of the granulocyte and/or granulocyte plus platelet depleted blood is completed the tube is removed and can be prepared for shipment.

The transfer device can be manufactured by any means known in the art including, but not limited to, standard plastic molding manufacturing or manufacture in a 3D printing device.

The following non-limiting examples will demonstrate the key attributes of the technology that will enable the transfer device disclosed herein to provide samples depleted of undesired cells such as granulocytes and platelets at a blood draw site prior to shipment of the sample to external sites for analysis. It cannot be over emphasized that the method and apparatus disclosed herein must be convenient and easy to use by trained laboratory personnel at the blood draw site. The following examples clearly demonstrate that this is the case for the invention disclosed herein. For the purpose of clarity only any device that starts with a tube of blood and ends with a tube ready to ship to an external site for analysis with granulocytes and/or platelets removed is anticipated by the invention disclosed herein.

### EXAMPLES

### 1. High Cell Recovery of Non-Targeted Cell Populations

It is absolutely essential that the technology used to deplete undesired cells prior to shipment to external sites yield the desired cells is close to 100% yield and the technology must work directly in undiluted whole blood at room temperature.

Whole blood was incubated with CD15 magnetic particles disclosed herein by the method disclosed herein to remove granulocytes. The depleted and control non-depleted samples were analyzed on a Becton Dickinson Flow Cytometer (Control; FIG. 1; Top). The analysis parameters were forward and 90-degree light scatter. The histogram shows 3 populations of cells: granulocytes (top), monocytes (middle) and lymphocytes (bottom). Following CD15 depletion (FIG. 1; bottom), the results clearly demonstrate effective removal of granulocytes with 100% recovery of the desired cells in this case lymphocytes. The slight depletion of monocytes is due to a subset of monocytes that express the CD15 antigen. These results clearly demonstrate the desired results using the magnetic particles disclosed herein: excellent depletion of a non-desired cell population (granulocytes) with quantitative recovery of a desired cell population (lymphocytes).

### 2. Rapid Removal of Granulocytes Using Anti-CD15 Metallic, Magnetic Particles

It is desirable that the binding of CD15 magnetic particles to granulocytes and the removal of the particle bound granulocytes by a magnetic field be as rapid as possible. The results disclosed in FIG. 2 demonstrate this is the case. Whole blood was incubated with CD15 magnetic particles disclosed herein by the method disclosed herein to remove granulocytes. The samples were analyzed on a Coulter 3-part differential hematology analyzer which yields lymphocytes (left peak), monocytes (middle peak) and granulocytes (right peak) as demonstrated in the control sample (FIG. 2; top left histogram). FIG. 2: top right, bottom left and bottom right demonstrate very rapid binding times with a magnetic separation time of 1 minute. These results demonstrate very rapid mixing times for granulocytes with the CD15 antigen on their cell surface. Experiments using the transfer device with a fixed volume of whole blood will determine the optimal mixing times and magnetic separation times for the optimal bead size, to be determined. It is clear from the results in FIG. 2 that very rapid mixing and magnetic separation times can be anticipated by laboratory personnel at the blood draw site.

### 3. Effect of CD15 Depletion on Various Cell Populations Present in Whole Blood

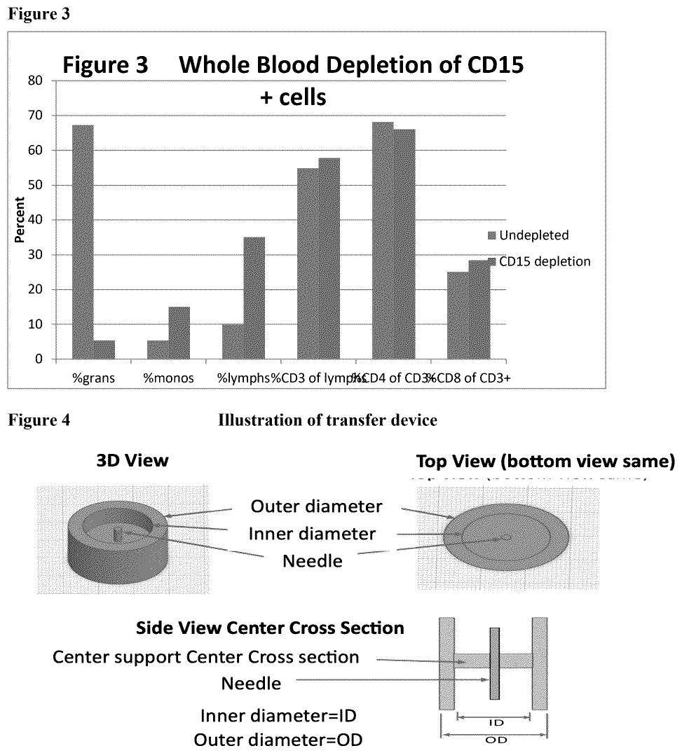

It is not only essential to remove granulocytes prior to shipment of blood to external sites but equally essential that following the depletion of CD15 positive cells using the magnetic particle separation technology described herein that the remaining PBMCs are not altered. Referring to FIG. 3 the removal of granulocytes did not adversely affect the following lymphocyte populations i.e. CD3, CD3/CD4 and CD3/CD8 populations. CD15 positive cells were removed by the method described herein and the whole blood was analyzed by dual color Flow Cytometry by procedures known in the art.

4. Depletion of specific cell types based on surface expressing antigens will also deplete extracellular vesicles (EV) expressing those antigens. For example, platelet EV will also be cleared when CD41 beads are used to deplete platelets. This would be a significant advantage in the field of “liquid biopsy” where circulating tumor EV are being detected as the platelet EV makeup a significant fraction of the total EV population. In fact, EV analysis is a rapidly rising field in itself and debulking EV is as important as debulking the cellular populations (10). Purification of exosomes, a subset of the EV, are accomplished by positive selection based on CD9, CD63, and CD81 from platelet free plasma. Platelets and platelet derived EV express CD9 and therefore even though the plasma is platelet free, significant fraction of the exosomes will be derived from platelets.

### CITED REFERENCES

Patents:

| | | |
|---|---|---|
| 5,262,302 | Russell | Nov. 16, 1993 |
| 5,576,185 | Coulter | Nov. 19, 1996 |
| 5,653,686 | Coulter | Aug. 5, 1997 |
| 9,435,799 | Russell | Sep. 6, 2016 |
| 9,739,768 | Russell | Aug. 23, 2017 |
| 5,466,574 | Liberti | Nov. 4, 1995 |
| 5,411,863 | Miltenyi | May 2, 1995 |
| 4,654,267 | Ugelstad | Mar. 31, 1987 |
| 4,707,523 | Chang | Nov. 17, 1987 |

### OTHER PUBLICATIONS

1. Bull M, Lee D, Stucky J, Chiu Y L, Rubin A, Horton H, et al. Defining blood processing parameters for optimal detection of cryopreserved antigen-specific responses for HIV vaccine trials. J Immunol Methods. 2007 Apr. 30; 322(1-2):57-69.
2. DeRose, R et al. Granulocyte contamination dramatically inhibits spot formation in AIDS virus-specific ELISpot assay. J. Immuno Methods. 2005 297; 177-186.
3. Kierstead L S, Dubey S, Meyer B, Tobery T W, Mogg R, Fernandez V R, et al. Enhanced rates and magnitude of immune responses detected against an HIV vaccine: effect of using an optimized process for isolating PBMC. AIDS Res Hum Retroviruses. 2007 January; 23(1):86-92.
4. Kim D W, Jang, Y Y et al. Overnight Storage of Blood in ACD Tubes at 4degC increases NK Cell Function in PBMC. Annals of Clinical and Lab Sciences. 2013; 43; 267.
5. Mallone, R, Mannering, S I et al. Isolation and preservation of peripheral blood mononuclear cells for analysis of islet antigen-reactive T cell responses: position statement of the T-Cell Workshop Committee of the Immunology of Diabetes Society. Clinical and Experimental Immunology. 2010; 163. 33-49
6. McKenna K C, Beatty K M, Vicetti Miguel R, Bilonick R A. Delayed processing of blood increases the frequency of activated CD11b+ CD15+ granulocytes which inhibit T cell function. Journal of Immunological Methods. [Comparative Study]. 2009 Feb. 28; 341(1-2):68-75.
7. Preobrazhensky, S N and Bahler, D W. Immunomagnetic bead separation of mononuclear cells from contaminating granulocytes in cryopreserved blood sample. Cryobiology 2009;59; 366-368
8. Miltenyi Catalog: Order no: 130-093-545
9. ThermoFisher Catolog: Catalog number: 11137D DYNAL CD15 magnetic beads
10. Zhao, Z.; Fan, J.; Hsu, Y. S.; Lyon, C. J.; Ning, B.; Hu, T. Y. Extracellular vesicles as cancer liquid biopsies: From discovery, validation, to clinical application. Lab Chip 2019, 19, 1114-1140.

We claim:

1. A device for the sterile depletion and transfer of granulocytes or granulocytes plus platelet depleted blood comprising:
a. a blood draw tube and septum cap under atmospheric pressure wherein liquid is contained in the tube under less pressure;
b. a bead tube containing antibody labeled magnetic particles capable of transferring the liquid from the blood draw tube under low pressure;
c. end-over-end mixer;
d. magnetic holder for separating the magnetic particles; and
e. while still in the magnetic field a second transfer device is used to transfer the blood depleted by the magnetic beads in to a new tube
f. shipping means.

2. The device in claim 1 wherein the liquid is undiluted whole blood.

3. The device in claim 1 wherein the antibody labeled magnetic particles are liquid or lyophilized.

4. The device in claim 1 wherein the antibody is a monoclonal antibody or polyclonal antibody.

5. The device in claim 1 wherein the bead tube is approximately 2 mm greater diameter than the blood draw tube's septum diameter.

6. The device in claim 1 wherein the bead is nickel.

7. The device in claim 1 wherein the density of the particles is approximately 4 to 10 g/cc or approximately around 9 g/cc.

8. The device in claim 1 wherein the low pressure blood draw is a puncture needle centered within the diameter of the top cap and extending from the bottom to allow full penetration through the septum cap to draw liquid from the blood tube into the bead tube.

9. The device in claim 1 wherein the low pressure transfer is through a luer-lock adaptor system.

10. The device in claim 1 wherein the magnetic holder is a Stemcell magnetic holder.

11. The device in claim 1 wherein the antibody labeled magnetic particles are CD15 magnetic particles or CD15 plus CD41/CD61 magnetic particles.

12. The device in claim 1 wherein the particles size range is from about 0.5 microns to about 3.5 microns.

13. A method for shipping granulocyte or granulocyte plus platelet depleted blood comprising:
a. obtaining a sample of undiluted whole blood in a blood draw tube and septum cap wherein a liquid is contained in the tube under reduced pressure..
b. transferring under low pressure the undiluted whole blood to a bead tube containing antibody labeled magnetic particles;
c. mixing by end-over-end rotation;
d. placing the bead tube in a magnetic holder to separate the magnetic particles;
e. transferring the blood depleted by the magnetic beads to a second transfer device while still in a magnetic field to a new tube; and
f. shipping the sample.

14. A method for shipping granulocyte or granulocyte plus platelet depleted blood comprising the device of claim 1.

15. The method in claim 14 wherein the liquid is undiluted whole blood.

16. The method in claim 14 wherein the antibody labeled magnetic particles are liquid or lyophilized.

17. The device in claim 14 wherein the antibody is a monoclonal antibody or polyclonal antibody.

18. The device in claim 14 wherein the bead tube is approximately 2 mm greater in diameter than the blood draw tube's septum diameter.

19. The device in claim 14 wherein the bead is nickel.

20. The device in claim 14 wherein the density of the particles is approximately 4 to 10 g/cc or approximately around 9 g/cc.

21. The device in claim 14 wherein the low pressure blood draw is a puncture needle centered within the diameter of the top cap and extending from the bottom to allow full penetration through the septum cap to draw liquid from the blood tube into the bead tube.

22. The device in claim 14 wherein the low pressure transfer is through a luer-lock adaptor system.

23. The device in claim 14 wherein the magnetic holder is a Stemcell magnetic holder.

24. The device in claim 14 wherein the antibody labeled magnetic particles are CD15 or CD15 plus CD41/CD61magnetic particles.

25. The device in claim 14 wherein the particles size range is from about 0.5 microns to about 3.5 microns.
